(19) United States
(12) Patent Application Publication
(10) Pub. No.: US 2021/0348189 A1
(43) Pub. Date: Nov. 11, 2021

(54) METHOD FOR FAST GENE EDITING AND CONSTRUCTING PRIMATE DISEASE MODEL

(71) Applicant: YONGXIANG ZHAO, Nanning, Guangxi (CN)

(72) Inventors: YONGXIANG ZHAO, Nanning, Guangxi (CN); LIPING ZHONG, Nanning, Guangxi (CN)

(21) Appl. No.: 17/277,731

(22) PCT Filed: Sep. 20, 2018
(86) PCT No.: PCT/CN2018/106658

§ 371 Date: Mar. 19, 2021

(51) Int. Cl. C12N 15/85; C12N 9/22; C12N 15/11; A01K 67/027; C12N 15/89

(52) U.S. Cl. CPC C12N 15/8509; C12N 9/22; C12N 15/111; A01K 67/0275; A01K 2267/03; C12N 2015/8527; C12N 2310/20; A01K 2227/106; C12N 15/89

(57) ABSTRACT

Provided is a primate disease model construction method based on fast gene edition, which including (a) constructing a sgRNA expression plasmid by using a gRNA oligonucleotide and a pX330 plasmid; (b) injecting the sgRNA expression plasmid prepared in step (a) into a hepatic portal vein of a primate animal by using a biopsy needle until liver cells become cancerous for obtaining a primate disease model. The sgRNA expression plasmid constructed by the gRNA oligonucleotide and pX330 plasmid can be directly injected into the primate liver tissue, so as to construct a tumor model rapidly.

### TECHNICAL FIELD

The present disclosure relates generally to a primate disease model filed, and more particularly, relates to a primate disease model construction method based on fast gene edition.

### BACKGROUND

The animal disease model construction is a necessary tool to study the pathogenesis, drug screening and vaccine development of the human tumor. Rodents, such as mice and rats, have become the most common model animals in the biomedical filed because of their advantages such as small size, rapid reproduction, clear genetic background and mature transgenic technology. However, the relationship between the rodent and human is relatively far, and the species difference is large, which limits the effectiveness of the rodent disease model in simulating the occurrence and development of human diseases. Compared with the mice and rats, non-human primates, such as cynomolgus monkey (Macaca fascicularis) and so on, are highly similar to human in the genetic evolution, nerve, physiology, immunity and gene sequence. Therefore, primates are the most valuable model animals to study the human gene function and diseases.

At present, the construction of mouse tumor model is mainly based on the gene targeting technology which is based on embryonic stem cells or somatic cell nuclear transplantation technology. It is realized by the transgenic animals having the precisely modified specific genes in which the target gene is knocked out or in.

However, for primates, it is difficult to obtain the animal disease model with the precisely modified specific genes by the similar technology strategy because the transplantation technologies of the embryonic stem cells and somatic cell nuclear are not mature. The traditional method is to construct the animal models by genetically modifying the germ cells. However, the mature time and reproductive cycle of the primate cynomolgus monkey are long, and the cost is high.

CRISPR-CAS9 system has attracted much attention as a new gene edition tool. It is mainly obtained by modifying an acquired immune system of bacteria. When exogenous DNA invades, CRISPR-RNA directs the CAS protein to perform a specific splicing and generates double strand breaks (DSBs) at the DNA target sites. DSB, which is generated after the DNA damage, repairs the damaged DNA for editing the genome at the fixed point, through activating two different intracellular intrinsic repair mechanisms of NHEJ (Non-homologous ending-joining) and HR (Homologous recombination).

However, in the somatic cell level inside the large primate animal, namely the cynomolgus monkey, which has the closest genetic background of human, the CRISPR technology is not available to construct the induced disease model.

### SUMMARY

The object of the present application is to provide a primate disease model construction method based on fast gene edition, aiming at the above problems mentioned in the prior art of the long cycle and high cost for constructing the primate tumor model.

In a first aspect, a primate disease model construction method based on fast gene edition, which is for non-diagnostic or non-therapeutic purpose, is provided. The primate disease model construction method based on fast gene edition comprises following steps:
(a) constructing a sgRNA expression plasmid by using a gRNA oligonucleotide and a pX330 plasmid;
(b) injecting the sgRNA expression plasmid prepared in step (a) into a hepatic portal vein of a primate animal by using a biopsy needle until liver cells become cancerous for obtaining a primate disease model.

Advantageously, the step (a) further comprises following steps:

(a1) splicing the pX330 plasmid with Bbs I restriction endonuclease and then recycling remains by a PCR clean recovery kit;

(a2) annealing single stranded gRNA oligonucleotide to form double stranded gRNA oligonucleotide and ligating the double stranded gRNA oligonucleotide with the pX330 plasmid which is linearized by the Bbs I restriction endonuclease, for obtaining the sgRNA expression plasmid.

Advantageously, the step (a) further comprises following steps:

(a3) culturing COS-7 cells with a DMEM medium;

(a4) taking out the COS-7 cells in a logarithmic phase for transfecting them with a Lipofectamine 3000 transfection agent, and then extracting genomic DNA according to operation steps of a genomic extraction kit after the transfection;

(a5) amplifying target sites of extracted genomic DNA by using Q5 enzyme, performing a T7E1 enzyme digestion detection and a TA clone sequencing, and then performing step (b) after confirming that a gene mutation has been generated at p53 gene target site sequence in the genomic DNA.

Advantageously, the step (a4) further comprises: placing the COS-7 cells in the logarithmic phase of growth into a six-well plate with 1.5×10⁶ cells in each well, transfecting them with the Lipofectamine 3000 transfection agent when a cell density reaches 70%, then harvesting the cells after 48 hours and extracting the genomic DNA according to the operation steps of the genomic extraction kit; wherein an amount of the sgRNA expression plasmid which is transfected by each well is controlled to between 2.5-3.0 μg.

Advantageously, the gRNA oligonucleotide comprises a base sequence of 5′-CAATTCTGCCCTCACAGCTC-3′.

Advantageously, the step (b) comprises:

(b1) under a guidance of B-mode ultrasound, positioning the biopsy needle at a sagittal part of a left branch of the hepatic portal vein, selecting a safe injection path, observing a needle tip position in real time after injecting the biopsy needle through abdominal skin, guiding the biopsy needle in front of a front wall of the sagittal part of the left branch of the hepatic portal vein, and then determining that the needle tip has successfully entered a portal vein lumen when there is a sense of breakthrough and pumped back blood;

(b2) injecting 120 ug pX330-p53-sgRNA or control plasmid pX330-EGFP-sgRNA with an injection volume of 400 μL into the portal vein lumen, rapidly;

(b3) injecting 1 ml 0.9% sodium chloride rapidly and then confirming that the CRISPR-Cas plasmid system is successfully injected through the portal vein injection by a bright hyperecho ultrasonography;

(b4) withdrawing the biopsy needle after the injection under a real-time ultrasound observation;

(b5) scanning the liver and its perihepatic areas by a ultrasound scan for eliminating bleeding and organ damage after all operations are completed.

Advantageously, the step (b5) further comprises amplifying the target site and purifying amplification fragments 45 days later, and performing a deep sequencing on the target site for analyzing a gene edition situation.

The primate disease model construction method based on fast gene edition and the sgRNA for specifically targeting the p53 tumor suppressor gene of the primate liver cell have the following beneficial effects. The sgRNA expression plasmid constructed by the gRNA oligonucleotide and pX330 plasmid can be directly injected into the primate liver tissue, so as to construct a tumor model rapidly.

### BRIEF DESCRIPTION OF THE DRAWINGS

FIG. 1 is a diagram showing the injection site of the pX330 plasmid and the gRNA.

FIG. 2 is a diagram showing a deep sequencing on the target site for analyzing the gene edition situation, 45 days after the liver puncture of the cynomolgus monkey.

### DETAILED DESCRIPTION OF THE PREFERRED EMBODIMENT

To make the object, the technical solution, and the advantage of the present application more clearly, the present application is further described in detail below with reference to the accompanying embodiments. It should be understood that the specific embodiments described herein are only used to explain the present disclosure and are not used to limit the same.

The primate disease model construction method based on fast gene edition of the present application is for non-diagnostic or non-therapeutic purpose and comprises following steps.

1. Constructing the sgRNA Expression Plasmid by Using the gRNA Oligonucleotide and pX330 Plasmid.

The gRNA oligonucleotide is designed according to the N20NGG design principle at a selected site of the first exon close to ATG. The gRNA oligonucleotide is single stranded and comprises a base sequence of 5′-CAATTCTGCCCTCACAGCTC-3′ (with a reverse sequence of 5′-GAGCTGTGAGGGCAG AATTG-3′). The above gRNA oligonucleotide is synthesized by SUZHOU GENEWIZ Inc. The pX330 plasmid expressing Cas9 protein and sgRNA is purchased from Addgene, Cambridge, Mass., USA.

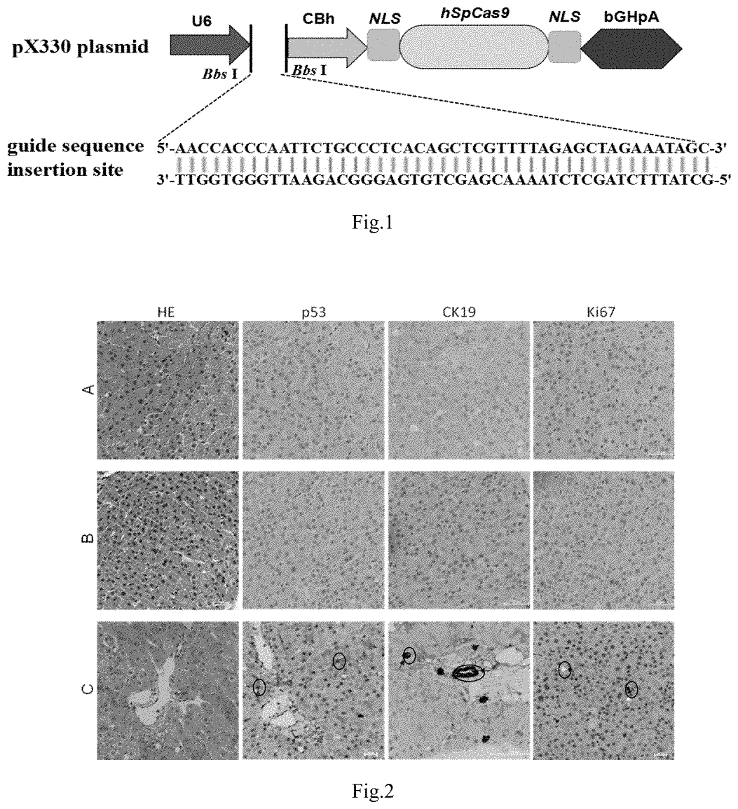

In this step, the pX330 plasmid is spliced with the Bbs I restriction endonuclease and then recycled by a PCR clean recovery kit which is purchased from Axygen Inc., USA. Then the single stranded gRNA oligonucleotide is annealed to form double stranded gRNA oligonucleotide for ligating the pX330 plasmid which is linearized by the Bbs I restriction endonuclease, for obtaining the sgRNA expression plasmid of pX330-p53-sgRNA. As shown in FIG. 1, U6 has initiated gRNA transcription, CBh promoter has initiated Cas9 protein expression, and NLS is a nuclear localization signal.

In this step, the same method can be used to construct the control plasmid of pX330-GFP-sgRNA for the GFP gene.

In order to verify whether the sgRNA expression plasmid pX330-p53-sgRNA is constructed successfully, the cell detection in vitro comprising following steps can be performed firstly.
(1) The COS-7 cells are cultured with a DMEM medium. The COS-7 cells are purchased from the cell bank of Shanghai Branch of Chinese Academy of Science. The cells are conventionally cultured in the DMEM medium (containing 10% fetal bovine serum, 100 U/ml penicillin and 100 μg/ml streptomycin) in a cell incubator at 37° C. containing 5% CO₂. The DMEM medium, fetal bovine serum and penicillin are purchased from GIBCO, Thermo Fisher Scientific Inc., Waltham, Mass., USA.
(2) The COS-7 cells in a logarithmic phase are taken out for transfecting them with the Lipofectamine 3000 transfection agent, and then the genomic DNA is extracted according to operation steps of the genomic extraction kit after the transfection. The Lipofectamine 3000 transfection agent is purchased from Invitrogen, Thermo Fisher Scientific Inc., Waltham, Mass., USA.

During the transfection, the COS-7 cells in the logarithmic phase are placed into a six-well plate with 1.5×10⁶ cells in each well. When the cell density reaches 70%, they are transfected with the Lipofectamine 3000 transfection agent. The amount of the sgRNA expression plasmid (pX330-p53-sgRNA) which is transfected by each well is controlled to between 2.5-3.0 μg. The pX330-gRNA-GFP carrier of the same amount is used as the negative control group. 48 hours after the transfection, the cells are harvested to extract genomic DNA according to the operation steps of the genomic extraction kit.
(3) Q5 enzyme is used for amplifying the target sites of extracted genomic DNA. T7E1 enzyme digestion detection and TA clone sequencing are performed. Then the following steps are further performed after confirming that a gene mutation has been generated at p53 gene target site sequence in the genomic DNA.

When amplifying target sites, the COS-7 genomic DNA is used as the template while the p53-F and p53-R are used as the primer, the reaction conditions are as follows: 98° C., 30 s; 35 cycles (98° C., 10 s; 60° C., 15 s; 72° C., 20 s), 72° C., 2 min; 95° C., 5 min; decreasing to 85° C. at a speed of −2° C./s; decreasing from 85° C. to 25° C. at a speed of −0.1° C./s. After the target site amplification, the product can be recycled by a PCR clean recovery kit. 10 μL recycled product is added into 0.5 μL T7E1 enzyme (purchased from New England Biolabs, USA) for enzyme digestion at 37° C. for 30 mins. The obtained product has been analyzed by 2% agarose gel electrophoresis. Q5 enzyme is used for amplifying the target site sequence to ligate the T carrier. The ligation product transforms the competent cell. Thirty monoclonal cells are randomly selected and sequenced. If there is a base insertion or deletion mutation in the target site of p53 gene in the COS cells, it indicates that there is a gene mutation.

2. Injecting the sgRNA expression plasmid prepared in step 1 into the hepatic portal vein of the primate animal by using a biopsy needle until the liver cells become cancerous. Then a primate disease model is obtained.

In this step, healthy male cynomolgus monkeys, aged from 5 to 8, weighing from 3.2-6.0 kg, can be selected and raised in the cynomolgus monkey medical application research base of Guangxi Fangchenggang Changchun Biotechnology Development Co., Ltd., China, which has been certified by AAALAC (Assessment And Accreditation Of Laboratory Animal Care). Under the guidance of the portable color doppler ultrasound instrument (Terason Co, MA, USA), the sgRNA expression plasmid of pX330-p53-sgRNA is injected into the hepatic portal vein of the cynomolgus monkey by the biopsy needle.

The specific steps are as follows. The cynomolgus monkeys are intramuscularly injected with shumianning II injection (0.1 ml/kg) and ethamsylate injection (0.1 g/each animal). After the successful anesthesia, the cynomolgus monkeys are fixed on the operating table in a horizontal position, shaved, disinfected with iodophor and covered with a towel. Under the guidance of B-mode ultrasound, the biopsy needle is positioned at the sagittal part of the left branch of the hepatic portal vein. A safe injection path is selected. Then the needle tip position is observed in real time after injecting the biopsy needle through the abdominal skin. The biopsy needle is guided in front of the front wall of the sagittal part of the left branch of the portal vein. When there is a sense of breakthrough and pumped back blood, it is determined that the needle tip has successfully entered a portal vein lumen. Then 120 ug pX330-p53-sgRNA or control plasmid pX330-EGFP-sgRNA with an injection volume of 400 μL, are injected into the portal vein lumen rapidly. Immediately, 1 ml 0.9% sodium chloride is rapidly injected. Then it is confirmed that the CRISPR-Cas plasmid system is successfully injected through the portal vein injection by a bright hyperecho ultrasonography. After the injection, the biopsy needle is withdrawn under the real-time ultrasound observation. After all operations are completed, the liver and its perihepatic areas are scanned by an ultrasound scan for eliminating the bleeding and organ damage

45 days after the liver puncture of the cynomolgus monkey by the sgRNA expression plasmid of pX330-p53-sgRNA, the target site is amplified, and the amplification fragments are purified. Then a deep sequencing is performed on the target site for analyzing the gene edition situation. The deep sequencing has showed that mutations are detected in the PAM region of the gRNA target site in 3 of the 6 cynomolgus monkeys in the experimental group, with a mutation rate of 50%. The mutations comprise the insertion and deletion of the nucleic acid sequence. The software analysis has showed that the length distribution of inserted or deleted nucleotide ranges from 1 bp to 20 bp, including one base deletion and insertion, and 20 bases deletion and insertion. According to the deep sequencing results, the InDel frequency at the p53Gene target site of the six cynomolgus monkeys in the experimental group has reached 5.39% at the highest.

As shown in FIG. 2, A is one representative of the healthy cynomolgus monkeys; B is one representative of the cynomolgus monkeys injected with the control plasmid of pX330-GFP-sgRNA, while C is one representative of the cynomolgus monkeys injected with the sgRNA expression plasmid of pX330-p53-sgRNA. The cynomolgus monkey C has produced the p53 positive cells, CK19 positive cells and Ki67 positive cells, as shown as the circles in the FIG. 2. The results has shown that the positive rates of p53 and Ki67 in the hepatoma cells and hepatic bile duct epithelial cells of the C cynomolgus monkey are significantly higher than those of the cynomolgus monkey B and A. The strong positive expression of CK19 in the hepatic bile duct epithelial cells of the C cynomolgus monkey is significantly higher than those of the cynomolgus monkey B and A.

2 months after the liver puncture of the cynomolgus monkey by the sgRNA expression plasmid of pX330-p53-sgRNA, the serum tumor markers AFP, CA125 and CA19-9 are significantly increased, and the liver cells and the hepatic bile duct epithelial cells show signs of transformation into malignant cells. These results suggest that liver cancer begins to form, which proves that the CRISPR-Cas9 system can perform a directly targeting edition to the p53 gene of the liver cell genome of the cynomolgus monkey in situ through the hepatic portal vein by the B-ultrasound minimally invasive intervention technology, thus causing the deletion mutation of the p53 tumor suppressor gene in the somatic cells, and rapidly inducing the construction of the liver cancer model.

The above is only a better specific embodiment of the present application, but the protection scope of the present application is not limited to this. Any change or replacement that can be easily thought of by a person familiar with the technical field within the technical scope of the present application shall be included in the protection scope of the present application. Therefore, the protection scope of the present application shall be subject to the protection scope of the claims.

1. A primate disease model construction method based on fast gene edition, for non-diagnostic or non-therapeutic purpose, comprising following steps:
(a) constructing a sgRNA expression plasmid by using a gRNA oligonucleotide and a pX330 plasmid;
(b) injecting the sgRNA expression plasmid prepared in step (a) into a hepatic portal vein of a primate animal by using a biopsy needle until liver cells become cancerous for obtaining a primate disease model.

2. The primate disease model construction method based on fast gene edition according to claim 1, wherein the step (a) further comprises following steps:
(a1) splicing the pX330 plasmid with Bbs I restriction endonuclease and then recycling remains by a PCR clean recovery kit;
(a2) annealing single stranded gRNA oligonucleotide to form double stranded gRNA oligonucleotide and ligating the double stranded gRNA oligonucleotide with the pX330 plasmid which is linearized by the Bbs I restriction endonuclease, for obtaining the sgRNA expression plasmid.

3. The primate disease model construction method based on fast gene edition according to claim 1, wherein the step (a) further comprises following steps:
(a3) culturing COS-7 cells with a DMEM medium;
(a4) taking out the COS-7 cells in a logarithmic phase for transfecting them with a Lipofectamine 3000 transfection agent, and then extracting genomic DNA according to operation steps of a genomic extraction kit after the transfection;
(a5) amplifying target sites of extracted genomic DNA by using Q5 enzyme, performing a T7E1 enzyme digestion detection and a TA clone sequencing, and then performing step (b) after confirming that a gene mutation has been generated at p53 gene target site sequence in the genomic DNA.

4. The primate disease model construction method based on fast gene edition according to claim 3, wherein the step (a4) further comprises: placing the COS-7 cells in the logarithmic phase into a six-well plate with 1.5×106 cells in each well, transfecting them with the Lipofectamine 3000 transfection agent when a cell density reaches 70%, then harvesting the cells after 48 hours and extracting the genomic DNA according to the operation steps of the genomic extraction kit; wherein an amount of the sgRNA expression plasmid which is transfected by each well is controlled to between 2.5-3.0 μg.

5. The primate disease model construction method based on fast gene edition according to claim 1, wherein the gRNA oligonucleotide comprises a base sequence of 5′-CAATTCTGCCCTCACAGCTC-3′.

6. The primate disease model construction method based on fast gene edition according to claim 1, wherein the step (b) comprises:
(b1) under a guidance of B-mode ultrasound, positioning the biopsy needle at a sagittal part of a left branch of the hepatic portal vein, selecting a safe injection path, observing a needle tip position in real time after injecting the biopsy needle through abdominal skin, guiding the biopsy needle in front of a front wall of the sagittal part of the left branch of the hepatic portal vein, and then determining that the needle tip has successfully entered a portal vein lumen when there is a sense of breakthrough and pumped back blood;
(b2) injecting 120 ug pX330-p53-sgRNA or control plasmid pX330-EGFP-sgRNA with an injection volume of 400 μL into the portal vein lumen, rapidly;
(b3) injecting 1 ml 0.9% sodium chloride rapidly and then confirming that the CRISPR-Cas plasmid system is successfully injected through the portal vein injection by a bright hyperecho ultrasonography;
(b4) withdrawing the biopsy needle after the injection under a real-time ultrasound observation;
(b5) scanning the liver and its perihepatic areas by a ultrasound scan for eliminating bleeding and organ damage after all operations are completed.

7. The primate disease model construction method based on fast gene edition according to claim 6, wherein the step (b5) further comprises amplifying the target site and purifying amplification fragments 45 days later, and performing a deep sequencing on the target site for analyzing a gene edition situation.

8. The primate disease model construction method based on fast gene edition according to claim 2, wherein the step (a) further comprises following steps:
(a3) culturing COS-7 cells with a DMEM medium;
(a4) taking out the COS-7 cells in a logarithmic phase for transfecting them with a Lipofectamine 3000 transfection agent, and then extracting genomic DNA according to operation steps of a genomic extraction kit after the transfection;
(a5) amplifying target sites of extracted genomic DNA by using Q5 enzyme, performing a T7E1 enzyme digestion detection and a TA clone sequencing, and then performing step (b) after confirming that a gene mutation has been generated at p53 gene target site sequence in the genomic DNA.

9. The primate disease model construction method based on fast gene edition according to claim 8, wherein the step (a4) further comprises: placing the COS-7 cells in the logarithmic phase into a six-well plate with 1.5×106 cells in each well, transfecting them with the Lipofectamine 3000 transfection agent when a cell density reaches 70%, then harvesting the cells after 48 hours and extracting the genomic DNA according to the operation steps of the genomic extraction kit; wherein an amount of the sgRNA expression plasmid which is transfected by each well is controlled to between 2.5-3.0 μg.
